(19) United States
(12) Patent Application Publication
(10) Pub. No.: US 2010/0160783 A1
(43) Pub. Date: Jun. 24, 2010

(54) ULTRASOUND IMAGING METHOD AND APPARATUS

(76) Inventors: Menachem Halmann, Bayside, WI (US); Hua Fan, Wuxi (CN); Huiren Chen, Wuxi (CN)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/641,995

(22) Filed: Dec. 18, 2009

(30) Foreign Application Priority Data
Dec. 19, 2008 (CN) 200810184425.7

(51) Int. Cl. A61B 8/14

(52) U.S. Cl. 600/447

(57) ABSTRACT

An ultrasound imaging method includes scanning an inspected object with a first cluster of ultrasonic beams having a first frequency and a first steer angle, to produce a first sub-frame which is used as a reference frame, and scanning the inspected object with a second cluster of ultrasonic beams having a second frequency different from the first frequency and a second steer angle different from the first steer angle, to produce a second sub-frame. The ultrasound imaging method also includes compounding the second sub-frame and the first sub-frame to form a compounded image, and displaying the compounded image.

### CROSS REFERENCE TO RELATED APPLICATIONS

This application claims the benefit of Chinese Patent Application No. 200810184425.7 filed Dec. 19, 2008, which is hereby incorporated by reference in its entirety.

### BACKGROUND OF THE INVENTION

The embodiments described herein relate to ultrasound imaging method and apparatus, in particular to ultrasound imaging method and apparatus using a technique for spatial and frequency compounding to obtain compounded ultrasound images.

The diasonograph is a kind of ultrasound imaging apparatus, which images the inner structure of a human body by using ultrasonic waves (hereinafter referred to as “ultrasound”) as information carriers, and the image information thereof corresponds to the real structure of the human body in respect of the spatial and time distribution. Medical ultrasound imaging establishes images by the echo (the reflected wave or backscattered wave of the ultrasound by the human tissues) of different sound strengths produced by the different sound characteristic impedance when the ultrasound propagating within the human body encounters different tissues and organs. It has been developed rapidly in recent years in imaging examinations for prognosis, invasive treatment and diagnosis because it is safe, reliable and cheap.

However, there are also some drawbacks in ultrasound imaging, such as poor image contrast and repeatability. Moreover, if the reflecting surface of the tissue in a human body is not smooth, and the roughness thereof corresponds to the wavelength of the incident ultrasonic wave, the echo signals produced by different reflecting sources may be either overlapped or counteracted due to their different phases, which is manifested by the granular sensation of the image and forms the so-called speckle noises. The speckle noises may cover some useful information in the image and interfere the doctor's diagnosis to some extent. Therefore, various methods have been adopted to increase the contrast of image, improve the specificity of diagnosis and increase the amount of information.

At present, the spatial compounding imaging technique has been used in the field of ultrasound imaging to reduce the speckle noises and meanwhile enhance the visualization of texture and boundaries. Spatial compounding is an imaging technique in which a number of ultrasound images of a given target that have been obtained from multiple angles are combined into a single compounded image. For example, the U.S. Pat. Nos. 6,126,599, 6,423,004 and 6,464,638 all describe the spatial compounding technique which is used in the processing of ultrasound imaging data to improve the imaging quality of the ultrasound image. In addition, the U.S. Pat. No. 6,733,458 describes a B-Steer technique applied in ultrasound guidance application, which slants transducer beams to a certain degree and employs spatial compounding to get better visual representation of invasive medical device, such as aspiration and biopsy needles, etc.

Although spatial compounding reduces the noise speckles and optimizes the imaging quality to a certain degree, it still has some deficiencies. For example, in the U.S. Patent Publication No. 2005/0124886 to James Jago et al. propose an ultrasound diagnostic imaging system and method which produces spatial compounded images by combining component image frames acquired from different viewing directions. Different regions of the spatial compounded images are composed of different numbers of overlapping component frames. As a result, the degree of spatial compounding varies in these regions. In this Publication, three frames of ultrasonic beams of substantially the same frequency are transmitted at a certain time sequence and steer angle to the inspected region, the echo signals reflected from the inspected region are received and are spatially compounded. Then such processing as temporal processing, spatial processing, frequency compounding or other types of processing compensating for variation in spatial compounding levels are performed to compensate for the spatial variation in spatial compounding due to the different number of overlapping component frames in various regions of the image. As a result, the variations in spatial compounding are compensated for to provide ultrasound images with more uniform speckle, noise, and temporal characteristics.

However, in said Publication, frequency compounding is an optional processing performed after spatial compounding. As shown in FIGS. 4a and 4b of said Publication, the received echo signals of substantially the same frequency are filtered by different filters and are split into several parts of different frequencies to compensate for the spatial variation in spatial compounding due to the different numbers of overlapping component frames in various regions of the image. So there is much complexity in implementation of this method.

The spatial compounding techniques in the prior art all combine successive image frames from different scan directions but with the same radiating/receiving frequency into one compounded image, and one common problem is that the selection of the steer angle of the ultrasonic beam is greatly limited by the transducer construction and emitting/receiving frequency, especially when happens to linear array probes with higher frequency and larger array element pitch. For example, in order to avoid the influence to the steered frame caused by the grating lobe, the steered frame can only use a limited steer angle to improve representation of the texture and layer of the tissue or invasive medical device, and this problem is more noticeable with transducers with larger array element pitch and higher frequency. In addition, the spatial compounding techniques in the prior art do not support to adjust transmitting frequency while constructing steered image frames. The current spatial compounding approach generally lacks flexibility.

In addition, for compounded images particularly used in guidance or navigation for invasive treatment, high image resolution and good continuity are necessary, which usually means that higher emitting/receiving frequency and greater steer angle will be appreciated. However, the available steer angles are limited by the acceptance angle of transducer array elements of the scanning device (e.g. probe), which in turn depends on the transducer array element pitch, frequency and construction method. Therefore, further measures should be taken to obtain ultrasound imaging of high imaging quality that has less speckle noises, high image resolution and good continuity.

### BRIEF DESCRIPTION OF THE INVENTION

The embodiments described herein provide an ultrasound imaging method and apparatus, which can obtain ultrasonic images of less speckle noises, high imaging quality and high flexibility.

According to the first aspect of the present invention, an ultrasound imaging method is provided, which comprises scanning an inspected object with a first cluster of ultrasonic beams having a first frequency and a first steer angle to produce a first sub-frame which is used as a reference frame; scanning with at least one cluster of ultrasonic beams having a frequency different from the first frequency and a steer angle different from the first steer angle to produce at least one other sub-frame; compounding said at least one other sub-frame and the first sub-frame to form a compounded image; and displaying said compounded image.

According to said ultrasound imaging method of the present invention, said at least one cluster of ultrasonic beams is steered right or left with respect to the first cluster of ultrasonic beams. The frequency of said at least one cluster of ultrasonic beams is user configurable according to the steer angle of the ultrasonic beams. Wherein the lager the steer angle of the ultrasonic beams is, the lower the frequency configured thereto is. In addition, the viewing field of the compounded image is the same as that of the reference frame.

According to said ultrasound imaging method of the present invention, said at least one cluster of ultrasonic beams is two clusters of ultrasonic beams that are steered right and left with respect to the first cluster of ultrasonic beams, respectively.

According to the second aspect of the present invention, an ultrasound imaging method is provided, which obtains more than two compounded images of more than two reference directions using the ultrasound imaging method according to the first aspect of the present invention; said more than two compounded images are compounded to produce an extended compounded image.

According to said ultrasound imaging method of the present invention, the viewing field of said extended compounded image is formed by combining the viewing fields of said more than two compounded images.

According to the third aspect of the present invention, an ultrasound imaging apparatus is provided, which captures a plurality of image signals of the same section through multiple scanning by the ultrasonic beams having different directions and different frequencies, and generates a compounded image according to said image signals and displays it. Said ultrasound imaging apparatus comprises: an image capturing unit, which captures a plurality of image signals through scanning by the ultrasonic beam having a reference direction and a first frequency and through one or multiple scanning by one or a plurality of ultrasonic beams having one or a plurality of directions different from the reference direction and a frequency different from the first frequency; an image processing unit which uses a plurality of image signals to produce a compounded image; and a display unit which displays the compounded image.

According to said ultrasound imaging apparatus, in the image capturing unit, said one or a plurality of ultrasonic beams having one or a plurality of directions different from the reference direction are steered right or left with respect to the reference direction. Moreover, the frequencies of said one or a plurality of ultrasonic beams are user configurable according to the steer angles of the ultrasonic beams. The larger the steer angles of said one or a plurality of ultrasonic beams, the lower the frequencies configured thereto are. In addition, the image processing unit can make the viewing field of the compounded image to be consistent with that of the image captured by scanning with the ultrasonic beam having the reference direction and the first frequency. Furthermore, said reference direction is variable.

According to the ultrasound imaging apparatus, the image capturing unit can capture a plurality of groups of image signals based on a plurality of reference directions, wherein each group of image signals includes said plurality of image signals captured based on one reference direction; said image processing unit generates a plurality of compounded images based on said plurality of groups of image signals, wherein each group of image signals generating one compounded image, and then combines said plurality of compounded images to produce an extended compounded image; said display unit displays the extended compounded image.

According to the ultrasound imaging apparatus, the viewing field of the compounded image generated by each group of image signals may be consistent with the viewing field of the image captured by scanning with the ultrasonic beam of the reference direction. The viewing field of said extended compounded image may be a combination of the viewing fields of said plurality of compounded images. Said display unit can display different viewing fields as different icons.

According to the ultrasound imaging apparatus, the image capturing unit comprises an emitting unit, an ultrasound probe, a receiving unit and a control unit, wherein the control unit controls the emitting unit to emit signals to drive the ultrasound probe and controls the receiving unit to receive the echo signals from the ultrasound probe; the image processing unit comprises a processing unit, a compounding unit, and a scan and conversion unit, wherein the processing unit processes the echo signals to produce image signals; the compounding unit compounds the produced image signals; and the scan and conversion unit scans and converts the compounded image data so as to be displayed. In addition, the image capturing unit may further comprise an operating unit for a user to enter instructions to the control unit.

According to the ultrasound imaging method and apparatus, the spatial and frequency compounding are performed. The ultrasound image frames viewed from multiple angles can be obtained by using the user configurable Tx/Rx frequency and beam steer angle. Then the compounded image having high resolution and good specular reflector delineation is formed through compounding. In the present invention, the frequency of the ultrasonic beam can be flexibly configured according to the steer angle, so it is possible to further enlarge the available range of beam steer angles, to improve texture appearance and lateral smoothness. Although the change of frequency sacrifices some resolution, it could well suppress the side lobe and grating lobe of the steered frame, reduces the speckle noises and clutters, and improves signal-to-noise ratio and the visibility of the inspected tissue and the needle. Experiments have proven that the image obtained by the spatial and frequency compounding of the present invention has better quality than the current spatial compounded image.

### BRIEF DESCRIPTION OF THE DRAWINGS

FIG. 1 illustrates the principle of linear scan;

FIG. 2 illustrates the principle of compounding scan;

FIG. 3 illustrates the method for generating a compounded image;

FIG. 4 is a block diagram of the ultrasound imaging apparatus, which is a preferred way of implementing the present invention;

FIG. 5 illustrates obtaining a two-dimensional compounded image using the spatial and frequency compounding technique of the present invention when the reference frame is not steered;

FIG. 6 illustrates obtaining a two-dimensional compounded image using the spatial and frequency compounding technique of the present invention when the reference frame is steered left;

FIG. 7 illustrates obtaining a two-dimensional compounded image using the spatial and frequency compounding technique of the present invention when the reference frame is steered right;

FIG. 8 illustrates the method of generating a compounded image;

FIG. 9 illustrates an example of displaying the icon of a compounded image.

### DETAILED DESCRIPTION OF THE INVENTION

The embodiments of the present invention will be described in detail below with reference to the figures.

Linear scan is used as an example to illustrate the present invention in the following, but the present invention is not limited to linear scan, it can also be used in sector scan, etc.

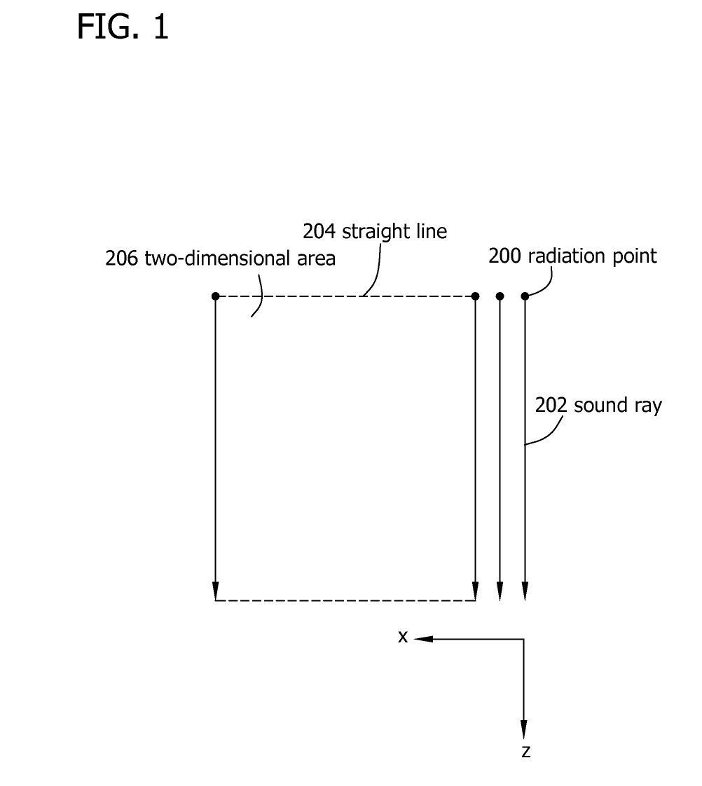

FIG. 1 illustrates the principle of scanning with a linear ultrasonic beam. As shown in FIG. 1, the scan is performed by a parallel excursion of a sound ray 202 along a straight line 204, said sound ray 202 is transmitted outside from a radiation point 200 in z direction, so that it sweeps a two-dimensional rectangular area 206 in x direction, thereby realizing linear scan. Wherein, the sound ray 202 corresponds to the central axis of the ultrasonic beam. By way of a parallel excursion of the aperture of the ultrasonic beam, scanning with the sound ray 202 in the scan direction is realized. By way of continuously changing the combination of a plurality of ultrasonic transducers when forming ultrasonic beams, moving of the aperture is realized, thereby realizing the scanning with the sound ray 202 in the scan direction.

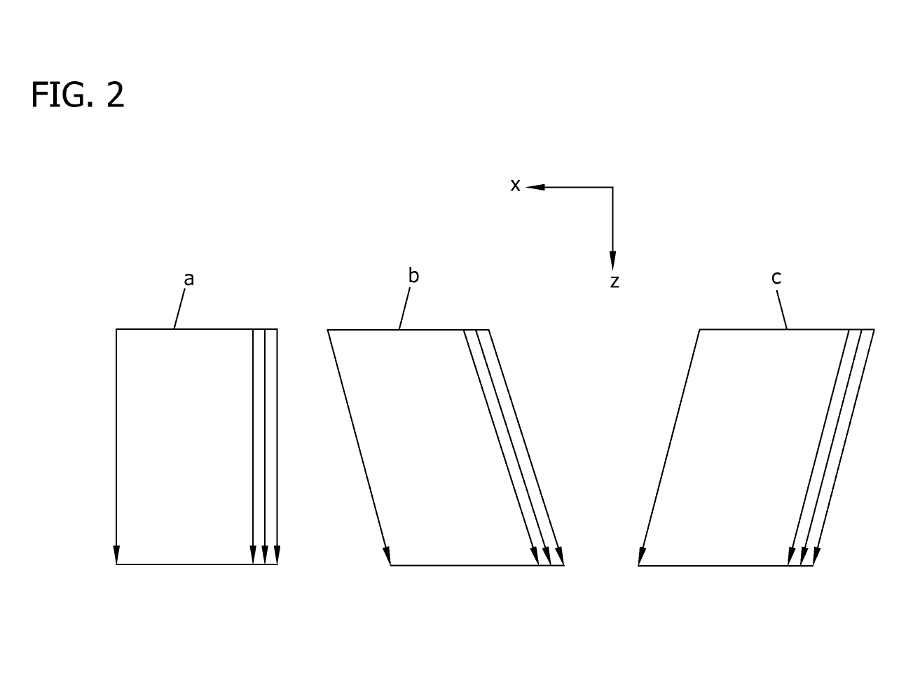

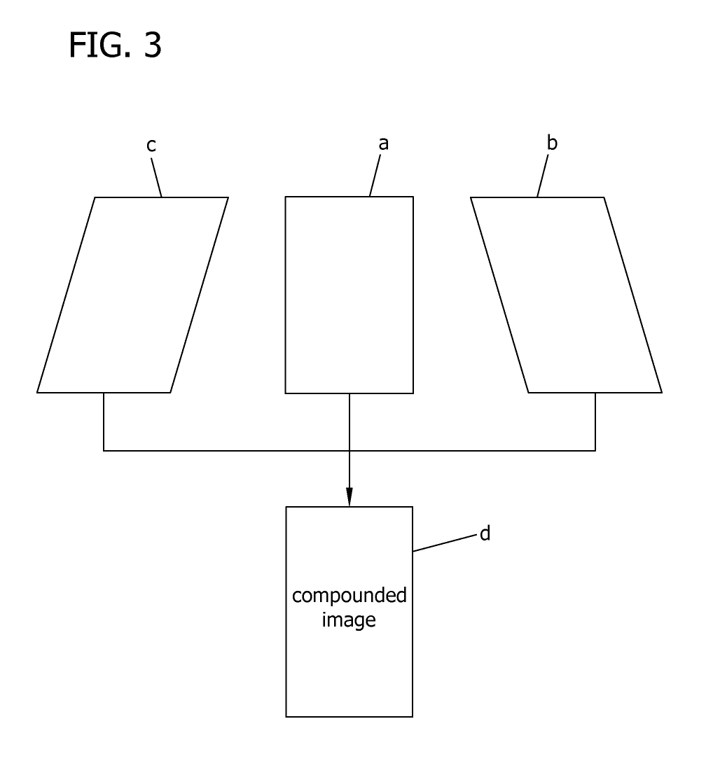

FIG. 2 and FIG. 3 depict the compounding method of the linear scan according to the present invention.

As shown in FIG. 2, there are three linear scan sub-frames for the compound scan. In the present invention, the sound rays or ultrasonic beams of the three sub-frames have different directions and frequencies, but said sub-frames are in the same cross-section that is to be imaged. The first sub-frame a is a sound ray scan frame having a first frequency and along the z direction. The second sub-frame b is a sound ray scan frame for oblique scan that has a second frequency and is steered right for a certain degree with respect to the z direction. The third sub-frame c is a sound ray scan frame for oblique scan that has a third frequency and is steered left for a certain degree with respect to the z direction.

FIG. 2 only shows three linear scan sub-frames for the compound scan, but in fact, the sub-frames may not be limited to three. The compound scan can be realized by a series of two scan sub-frames or a series of four scan sub-frames or more linear scan sub-frames. The compound scan including a series of three scan sub-frames is used as an example in the following, while it is applicable to other situations likewise.

In the linear scan, the density of the sound ray is uniform on the frames from the near field to the far field. That is, with respect to sub-frame a, the density of the sound ray in the z direction is uniform on said sub-frame; with respect to sub-frame b, the density of the sound ray for scan steered to the right is uniform on said sub-frame; and with respect to sub-frame c, the density of the sound ray for scan steered to the left is uniform on said sub-frame.

FIG. 3 illustrates the generation of a compounded image from the three scan sub-frames as shown in FIG. 2. As shown in FIG. 3, a compounded image d is generated by compounding the images of the three sub-frames a, b and c. Only the part that is of the same shape as sub-frame a is taken during the compounding, while the parts of sub-frames b and c that do not overlap with sub-frame a are not taken into account. The compounding of the frame images will be named frame compounding in the following. In the compounded image d, the image of the real echo source is enhanced by superposing, while the random signal components, such as noises and spikes, are counteracted by superposing, so the random noises of the images of the real echo source are smoothed and suppressed, so that the compounded image can have clearer texture and boundary information, and the compounded image d of better quality is obtained.

The present invention provides an ultrasound imaging method and apparatus based on the above principles and methods as illustrated in FIGS. 1, 2 and 3, which solves, to some extent, the problem that the steer angle is limited in ultrasound scan. Specifically, in the ultrasound imaging method of the present invention, a first sub-frame is formed by transmitting and receiving a first cluster of ultrasonic beams having a first steer angle and a first frequency and is used as a reference frame; two or more sub-frames are formed by transmitting and receiving other two or more clusters of ultrasonic beams that are steered with respect to the reference frame, wherein said two or more clusters of ultrasonic beams having the same or different frequencies, but different from the first frequency. The two or more sub-frames are compounded with the reference frame to form a single compounded image. Said two or more clusters of ultrasonic beams are steered right or left with respect to the first cluster of ultrasonic beams. When there are two other clusters of ultrasonic beams, it is preferable that one cluster is steered right and the other is steered left. The present invention extends the steer angle by configuring the corresponding Tx/Rx frequencies to the ultrasonic beams of different scan directions. The frequencies configured to the ultrasonic beams are reduced with the increase of the steer angle. For example, when the reference frame is a scan frame without being steered, the steer angle is extended by reducing the Tx/Rx frequency of the slanted ultrasonic beams. Moreover, the steer angles of the two or more clusters of ultrasonic beams are user-configurable. In addition, the viewing field of the compounded image obtained through the above method of the present invention is the same as the viewing field of the reference frame.

Furthermore, in the present invention, the above method can also be used to obtain compounded images of multiple reference directions, then the compounded images of multiple reference directions are further compounded to generate an extended compounded image. The viewing field of said extended compounded image is formed by combining the viewing fields of said more than two compounded images.

The ultrasound imaging apparatus according to the present invention comprises an image capturing unit for capturing a plurality of image signals through scanning with an ultrasonic beam having a reference direction and a first frequency and through one or a plurality of times of scanning with one or more ultrasonic beams having one or more directions different from the reference direction and frequencies different from the first frequency; an image processing unit for generating a compounded image using a plurality of image signals, and a display unit for displaying the compounded image.

In a embodiment of the ultrasound imaging apparatus according to the present invention, the image capturing unit comprises an emitting unit, an ultrasound probe, a receiving unit, and a control unit; the image processing unit comprises a processing unit, a compounding unit, and a scan and conversion unit. Of course, the combination of these modules is not limited to the embodiment, but other ways of combination can be adopted according to the needs.

In addition, the ultrasound imaging apparatus of the present invention may further comprise an operating unit for entering instructions to the control unit so as to make the control unit to perform the corresponding operations.

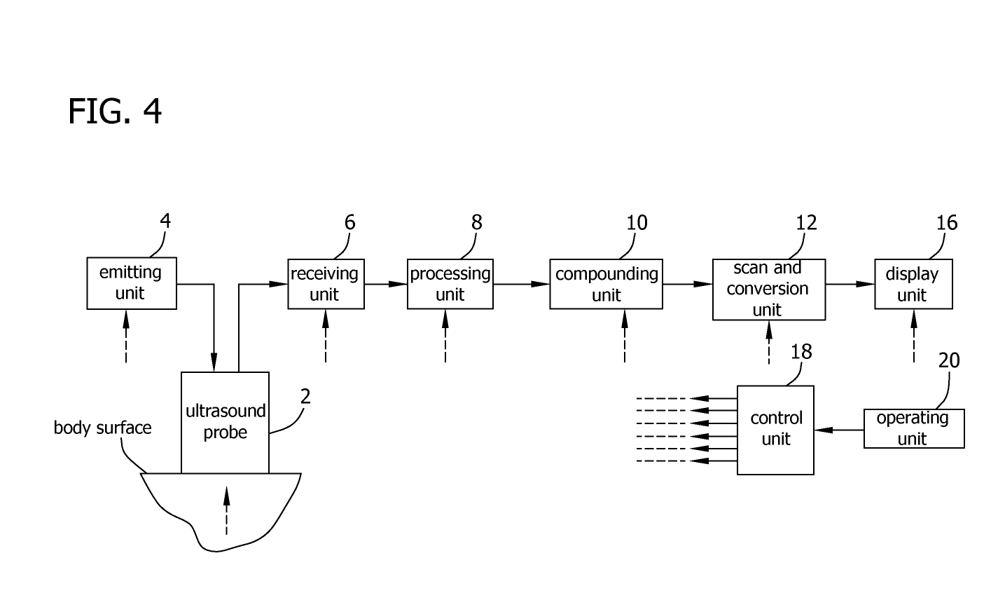

FIG. 4 is a block diagram of a embodiment of the ultrasound imaging apparatus according to the present invention. As shown in FIG. 4, said apparatus comprises an ultrasound probe 2, an emitting unit 4, a receiving unit 6, a processing unit 8, a compounding unit 10, a scan and conversion unit 12, a display unit 16, a control unit 18 and an operating unit 20. The ultrasound probe 2 contacts the body surface when being in use. The ultrasound probe 2 has an ultrasound transducer array and each individual ultrasound transducer is made of piezoelectric material, such as PZT (plumbum (Pb) zirconate (Zr) titanium (Ti)) ceramic, etc. The operator sends instructions by the operating unit 20 to the control unit 18, such that the control unit 18 controls the emitting unit 4 to emit signals to drive the ultrasound probe 2 which generates ultrasonic beams to scan the inspected part. The receiving unit 6 receives from the ultrasound probe 2 the echo signals and performs such processing as amplification, then said signals are sent to the processing unit 8. The processing unit 8 detects the input signals to generate image information. The compounding unit 10 compounds image information of different directions, then said image information is scanned and converted by the scan and conversion unit 12 to form the scanned and converted image data which are finally displayed on the display unit 16. The processing unit 8 can be a B-steer processing unit.

The emitting unit 4, receiving unit 6, processing unit 8, compounding unit 10, scan and conversion unit 10 and display unit 16 are all controlled by the control unit 18. Said control unit 18 controls the emitting unit 4 to emit a scan frame of a certain frequency and a certain direction, and controls the receiving unit 6 to receive the echo signal of the scan frame with the corresponding frequency and direction, then controls the echo signal to be processed in the processing unit 8, to be compounded properly or as desired in the compounding unit 10, to be scanned and converted in the scan and conversion unit 12, and finally, controls the compounded image to be displayed on the display unit 16. The control unit 18 can be, for example, a computer, etc. Usually, the user enters the operation instructions into the control unit 18 via the operating unit 20 to realize the corresponding control. The operating unit 20 can be, for example, a keyboard, a track ball, etc.

Several preferred embodiments of the present invention will be described below to make the present invention clearer.

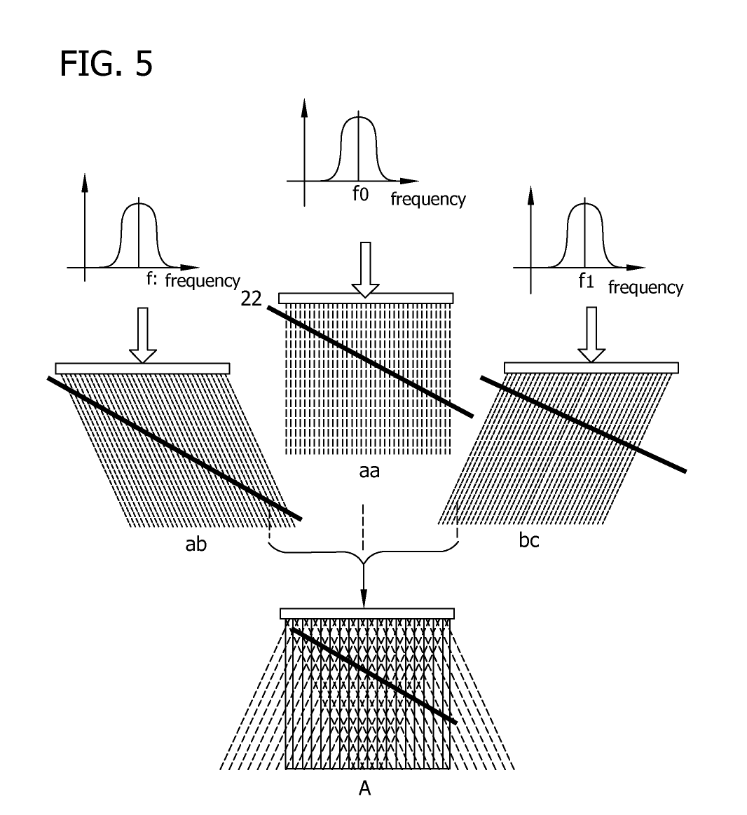

FIG. 5 shows an exemplary embodiment according to the present invention. As shown in FIG. 5, there is no steer in the direction of the sound ray of the reference frame. The first sub-frame aa as the reference frame has sound ray beams of a first frequency, which are a square from the front; the second sub-frame ab has sound ray beams of a second frequency, which are steered right with respect to the direction of the sound ray of the first sub-frame; the third sub-frame be has sound ray beams of a third frequency, which are steered left with respect to the direction of the sound ray of the first sub-frame.

The first sub-frame aa, second sub-frame ab and third sub-frame ac are respectively generated by the control unit 18 controlling the emitting unit 4 to emit signals to drive the ultrasound probe 2. Information of three images is obtained, via the receiving unit 6 and processing unit 8, from the echo signals generated by scanning the inspected area as shown in FIG. 4. Then the compounding unit 10 compounds the obtained information of the three images to generate a compounded image A. The compounded image A can be set to have the same shape and direction as the first frame aa that is used as the reference frame and its sides are not slanted, i.e. being a rectangle. The compounded image A is named frame A for short in the following. Said compounded image A is displayed on the display unit 16 after being scanned and converted by the scan and conversion unit 12.

Table 1 shows an example of the configuration of the steer angle and the corresponding Tx/Rx frequency of the two-dimensional image as shown in FIG. 5

TABLE 1

| | |
|---|---|
| Steer | Emitting frequency |

| angle | f0 = 12 MHz | f1 = 10 MHz |
|---|---|---|
| 0° | ✓ | |
| −15° | | ✓ |
| 15° | | ✓ |

According to table 1, the first sub-frame aa of FIG. 5 uses f0=12 MHz as the Tx/Rx frequency; the second sub-frame ab and the third sub-frame ac respectively are steered right and left for 15° with respect to the first sub-frame aa, and they both use f1=10 MHz as the Tx/Rx frequency.

The above table 1 gives an example of the configuration of a high-frequency no-steered scan sub-frame (used as the reference frame) and the configuration of two lower-frequency scan sub-frames having the same steering. However, the present invention is not limited to such an example, for instance, the steer angles of the two steered frames may be different and the frequencies thereof may also be different. Moreover, the present invention is not limited to using two steered frames, it can use three or more steered frames.

The frame A obtained by spatial and frequency compounding can increase the slant angle of the steered frame to make the sound ray thereof and needle 22 intersect at an angle close to right angle, such that frame A is more clearly visualized in texture and boundary, and this enables frame A to be suitable for tracking the needle 22 stuck into the body towards the lower right to form an included angle with the body surface in the FOV. Meanwhile, since higher frequency is configured to the non-steered frame, the compounded image can maintain a high resolution performance.

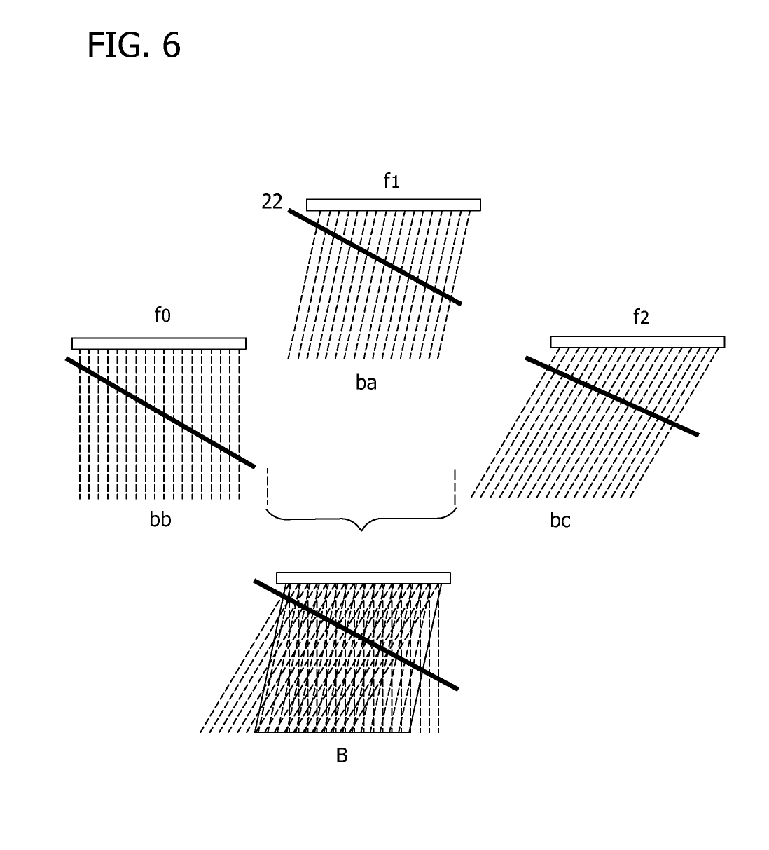

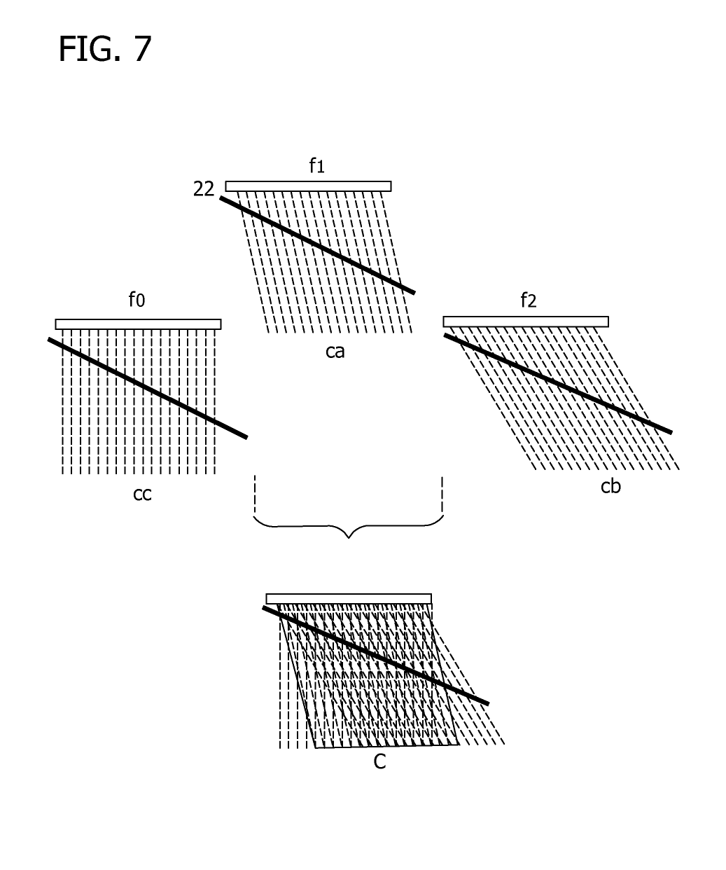

Based on the same principle as illustrated by FIG. 5, steered frames with different slanted angles can employ relevant Tx/Rx frequencies to enlarge steer angle for the purpose of good representation of invasive medical device. Now the reference direction of the sound ray of the first sub-frame aa in FIG. 5 can be selectively changed by the user according to the need. When the reference direction of the sound ray of the first sub-frame aa is changed, the direction of the sound ray of the second sub-frame ab and the direction of the sound ray of the third sub-frame ac are correspondingly changed. In this case, the compounded image can be formed by such sub-frames: one reference frame which is steered right or left at a certain degree, and two or more steered sub-frames which are steered right or left with respect to the reference frame. FIGS. 6 and 7 illustrate the process of obtaining the compounded image with the reference frame steered to different directions.

FIG. 6 depicts the obtaining of a two-dimensional compounded image by means of the spatial and frequency compounding technique according to the present invention with the reference frame steered left. As shown in FIG. 6, the needle 22 is stuck into the body towards the lower right as shown by the arrow, forming an included angle with the body surface. According to the present invention, a first sub-frame ba is obtained by scanning with the sound ray of a first frequency f1, which is steered to the left, and taking the direction of the sound ray of the first sub-frame ba as the reference direction of the sound ray; a second sub-frame bb is obtained by scanning with the sound ray of a second frequency f0, which is steered to the right with respect to the direction of the sound ray of the first sub-frame ba; and a third sub-frame bc is obtained by scanning with the sound ray of a third frequency f2, which is steered to the left with respect to the direction of the sound ray of the first sub-frame ba.

The first sub-frame ba, second sub-frame bb and third sub-frame bc are respectively generated by the control unit 18 controlling the emitting unit 4 to emit signals to drive the ultrasound probe 2. Information of three images is obtained, via the receiving unit 6 and processing unit 8, from the echo signals generated by scanning the inspected area, as shown in FIG. 4. Then the compounding unit 10 compounds the obtained information of the three images to generate a compounded image B. The frame of the compounded image B can be set to have the same shape and direction as the first sub-frame ba, i.e. to be a parallelogram having its sides slanted to the left. The compounded image B is named frame B for short in the following. Said compounded image B is displayed on the display unit 16 after being scanned and converted by the scan and conversion unit 12.

It can be found from a comparison between the frame A in FIG. 5 and the frame B in FIG. 6 that the angle between the sound ray of frame B and the needle 22 is more closer to a right angle than the angle between the sound ray of frame A and the needle 22, so frame B can make the image of needle 22 more visible. Therefore, said frame B that offsets to the left in the far field is more suitable for tracking the needle 22 stuck into the body towards the lower right to form an included angle with the body surface in the FOV and is suitable to be used as a guidance or navigation image.

FIG. 7 depicts the obtaining of a two-dimensional compounded image by means of the spatial and frequency compounding technique according to the present invention with the reference frame steered right. As shown in FIG. 7, the needle 22 is stuck into the body towards the lower right as shown by the arrow, forming an included angle with the body surface. According to the present invention, a first sub-frame ca is obtained by scanning with the sound ray of a first frequency f1, which is steered to the right, and taking the direction of the sound ray of the first sub-frame ca as the reference direction of the sound ray; a second sub-frame cb is obtained by scanning with the sound ray of a second frequency f2, which is steered to the right with respect to the direction of the sound ray of the first sub-frame; and a third sub-frame cc is obtained by scanning with the sound ray of a third frequency f0, which is steered to the left with respect to the direction of the sound ray of the first sub-frame.

The first sub-frame ca, second sub-frame cb and third sub-frame cc are respectively generated by the control unit 18 controlling the emitting unit 4 to emit signals to drive the ultrasound probe 2. Information of three images is obtained, via the receiving unit 6 and processing unit 8, from the echo signals generated by scanning the inspected area. Then the compounding unit 10 compounds the obtained information of the three images to generate a compounded image C. The frame of the compounded image C can be set to have the same shape and direction as the first sub-frame ca, i.e. to be a parallelogram having its sides slanted to the right. The compounded image C is named frame C for short in the following. Said compounded image C is displayed on the display unit 16 after being scanned and converted by the scan and conversion unit 12.

It can be found from a comparison between the frame C in FIG. 7 and the frame A in FIG. 5 and frame B in FIG. 6 that the direction of frame C corresponds to the direction of the needle 22 stuck into the body towards the lower right to form an included angle with the body surface in the FOV, so it is the least suitable for tracking the needle 22. Therefore, when the needle 22 sticks into the body towards the lower right to form an included angle with the body surface, it is preferable to use frame B instead of frame C to track the needle 22.

Data in the following table 2 are used as an example to illustrate the configuration of the steer angles and the corresponding frequencies in FIGS. 6 and 7.

TABLE 2

| Steer | Emitting frequency | | |
|---|---|---|---|
| angle | f0 = 12 MHz | f1 = 10 MHz | f2 = 8 MHz |
| 0° | ✓ | | |
| −15° | | ✓ | |
| 15° | | ✓ | |
| −30° | | | ✓ |
| 30° | | | ✓ |

In the situation as shown in FIG. 6, the non-steered sub-frame bb uses a higher frequency f0=12 MHz, the slanted sub-frame be having a maximum left-steered angle of 30° uses the lowest frequency F2=8 MHz, and the reference frame ba steered left for 15° uses an middle frequency f1=10 MHz. In the situation shown in FIG. 7, the non-steered sub-frame cc uses a highest frequency f0=12 MHz, the slanted sub-frame cb having a maximum right-steered angle of 30° uses the lowest frequency F2=8 MHz, and the reference frame ca steered right for 15° uses an middle frequency f1=10 MHz. Through the above configuration of the adjustable frequency for each sub-frame, the compounded image having the same steer angle as the reference frame can use steer angles of a wider range to obtain the compounded image of a desired performance.

It can be seen from the above that when performing invasive treatment on the patients, the user may send proper instructions to the control unit 18 via the operating unit 20, and adopt the ways shown in FIGS. 5, 6 and 7 to obtain the compounded image to be used as the guidance or navigation image, thereby making it more convenient to use the ultrasound probe.

Further, in order to improve the convenience of using the ultrasound probe, according to the present invention, compounded images of different FOV can be effectively obtained by properly combining frames A, B and C, and this enables the user to adjust the viewing field (FOV) of the compounded image via the operating unit 20 according to the direction along which the invasive medical device is applied.

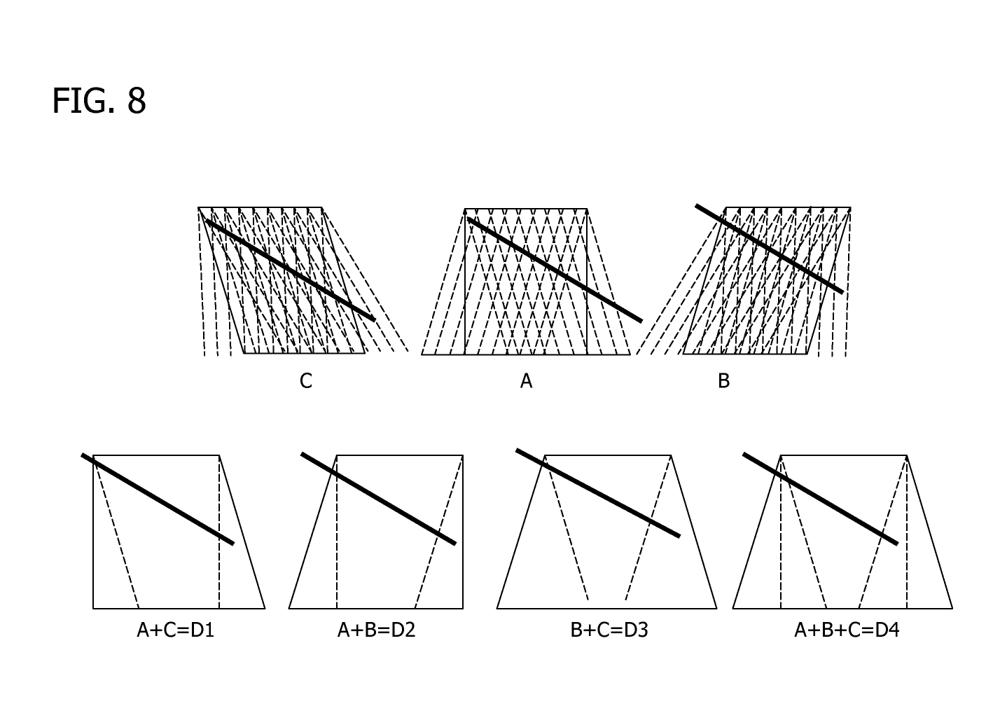

FIG. 8 illustrates an example of combining frames A, B and C. As shown in FIG. 8, the compounded image D1 is produced by combining frames A and C. One side of the frame of the compounded image D1 is slanted to the right. In the obtained compounded image D1, the overlapping portion of compounded images A and C has improved image quality, and it is clear that the compounded image D1 has an extended FOV as compared to the compounded images A and B.

As shown in FIG. 8, the compounded image D2 is produced by combining frames A and B. One side of the frame of the compounded image D2 is slanted to the left. In the obtained compounded image D2, the overlapping portion of the compounded images A and B has improved image quality, and it is clear that the compounded image D2 has an extended FOV as compared to the compounded images A and B.

As shown in FIG. 8, the compounded image D3 is produced by combining frames B and C. The frame of the compounded image D3 is in the form of a trapezium with both sides slanted. In the obtained compounded image D3, the overlapping portion of the compounded images B and C has improved image quality, and it is clear that compounded image D3 has an extended FOV as compared to the compounded images B and C.

As shown in FIG. 8, the compounded image D4 is produced by combining frames A, B and C. The compounded image D4 is in the form of a trapezium with both sides slanted. In the obtained compounded image D4, the overlapping portion of the compounded images A, B and C has the best image quality, and the overlapping portion of the compounded images A and B and the overlapping portion of the compounded images B and C have better image quality. Besides, it is clear that the compounded image D4 has an extended FOV as compared to the compounded images A, B and C.

The compounded images D1, D2, D3 and D4 are extended compounded images, which are produced by the compounding unit 10. Said compounded images D1, D2, D3 and D4 are applied to guidance and navigation that require higher image quality and larger FOV.

In the above embodiments of the present invention, frame buffers can be provided at appropriate positions to store the corresponding intermediate frame or compounded frame according to the needs, for example, frame buffers can be arranged in front of and behind the compounding unit 10.

The above embodiment describes the circumstance where the needle 22 sticks into the body towards the lower right to form an included angle with the body surface. Besides the needle 22 can also stick towards the lower left. When it sticks into the body towards the lower left, the compounded images C, D1, D3 and D4 are images suitable for tracking the FOV of the needle that sticks in said direction.

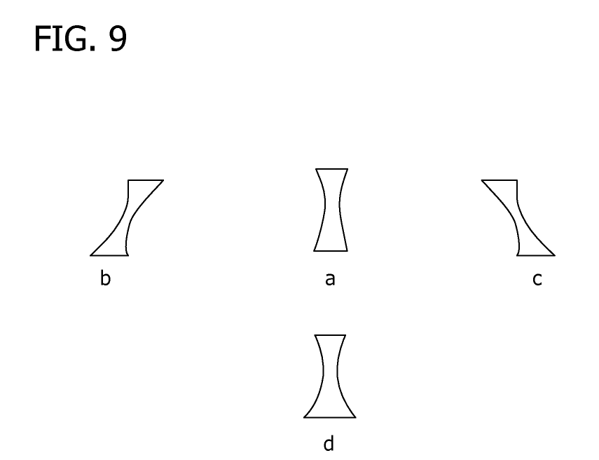

FIG. 9 depicts an example of the icons displayed on the display unit 16 corresponding to the frames A, B, C and D, which enables the user to easily identify the type of the displayed compounded image. As shown in FIG. 9, each of the frames is represented by a corresponding icon of a bowknot shape. Specifically, frame A is represented by icon a having the shape of a upstanding bowknot whose top side and bottom side are of the same length; frame B is represented by icon b having the shape of a bowknot that inclines to the lower left; frame C is represented by icon c having the shape of a bowknot that inclines to the lower right; and frame D (including D1, D2, D3 and D4) is represented by icon d having the shape of a upstanding bowknot whose top side is shorter than the bottom side. These icons can also be displayed on the touch panel of the operating unit 20 for selecting the type of compounding scan. When using said icons to select the type of compounding scan, the reference direction of the sound ray in respective frame can be set to a predefined default, thus the way of operating the device is simplified.

The above are only the preferred embodiments of the present invention, while it should be pointed out that to those skilled in the art, many improvements, modifications and variations can be made without departing from the spirit of the invention, so all such improvements, modifications and variations should be considered as falling within the scope of protection of this application.

1. An ultrasound imaging method, comprising:
scanning an inspected object with a first cluster of ultrasonic beams having a first frequency and a first steer angle, to produce a first sub-frame which is used as a reference frame;
scanning the inspected object with a second cluster of ultrasonic beams having a second frequency different from the first frequency and a second steer angle different from the first steer angle, to produce a second sub-frame;
compounding the second sub-frame and the first sub-frame to form a compounded image; and
displaying the compounded image.

2. The ultrasound imaging method of claim 1, further comprising:
scanning the inspected object with a third cluster of ultrasonic beams having a third frequency different from the first frequency and a third steer angle different from the first steer angle, for generating a third sub-frame.

3. The ultrasound imaging method of claim 1, wherein scanning the inspected object with a second cluster of ultrasonic beams comprises scanning with the second cluster of ultrasonic beams that is steered left or right with respect to the first cluster of ultrasonic beams.

4. The ultrasound imaging method of claim 1, wherein the second frequency of the second cluster of ultrasonic beams is user-configurable according to the second steer angle of the second cluster of ultrasonic beams.

5. The ultrasound imaging method of claim 4, wherein a size of the second steer angle is inversely related to the second frequency.

6. The ultrasound imaging ultrasound imaging method of claim 1, wherein a viewing field of the compounded image is the same as a viewing field of the reference frame.

7. An ultrasound imaging method, comprising:
scanning an inspected object with a first cluster of ultrasonic beams having a first frequency and a first steer angle, to produce a first sub-frame which is used as a reference frame;
scanning the inspected object with a second cluster of ultrasonic beams having a second frequency different from the first frequency and a second steer angle different from the first steer angle, to produce a second sub-frame;
scanning the inspected object with a third cluster of ultrasonic beams having a third frequency different from the first frequency and a third steer angle different from the first steer angle, for generating a third sub-frame;
compounding at least two of the first sub-frame, the second sub-frame, and the third sub-frame to form a first compounded image;
compounding at least two of the first sub-frame, the second sub-frame, and the third sub-frame to form a second compounded image; and
compounding the first compounded image and the second compounded image to generate an extended compounded image.

8. The ultrasound imaging method of claim 7, wherein a viewing field of the extended compounded image is formed by combining a respective viewing field of each of the first compounded image and the second compounded image.

9. An ultrasound imaging apparatus configured to capture a plurality of image signals of a scanned section through multiple scanning by a plurality of ultrasonic beams having different directions and different frequencies, said ultrasound imaging apparatus further configured to generate a compounded image according to the plurality of image signals and to display the compounded image, said ultrasound imaging apparatus comprising:
an image capturing unit configured to capture the plurality of image signals using a first scan with a first plurality of ultrasonic beams having a reference direction and a first frequency and using a second scan with a second plurality of ultrasonic beams having a second direction different from the reference direction and a second frequency different from the first frequency;
an image processing unit configured to produce a first compounded image based on the plurality of imaging signals; and
a display unit configured to display the first compounded image.

10. The ultrasound imaging apparatus of claim 9, wherein said image capturing unit is configured to steer the second plurality of ultrasonic beams right or left with respect to the reference direction.

11. The ultrasound imaging apparatus of claim 9, wherein the second frequency of the second plurality of ultrasonic beams is user-configurable according to the second steer angle.

12. The ultrasound imaging apparatus of claim 11, wherein a size of the second steer angle of is inversely related to the second frequency.

13. The ultrasound imaging apparatus of claim 9, wherein said image processing unit is configured to set a viewing field of the first compounded image to be consistent with a viewing field of a first image captured by scanning with the first plurality of ultrasonic beams having the reference direction and the first frequency.

14. The ultrasound imaging apparatus of claim 9, wherein in said image capturing unit, the reference direction is variable.

15. The ultrasound imaging apparatus of claim 9, wherein
said image capturing unit is configured to capture a third plurality of image signals using a third scan with a third plurality of ultrasonic beams having a third direction different than the reference direction and a third frequency different than the first frequency;
said image processing unit is configured to generate a second compounded image based on at least two of the first, second, and third plurality of image signals, wherein each plurality of image signals is used to generate a related compounded image, said image processing unit further configured to combine the plurality of compounded images to produce an extended compounded image;
said display unit configured to display the extended compounded image.

16. The ultrasound imaging apparatus of claim 15, wherein a respective viewing field of the compounded image generated by each group of image signals is consistent with the viewing field of the image captured by scanning with the ultrasonic beams of the reference direction.

17. The ultrasound imaging apparatus of claim 16, wherein the viewing field of the extended compounded image is a combination of the viewing fields of the plurality of compounded images.

18. The ultrasound imaging apparatus of claim 13, wherein said display unit is configured to display icons of different viewing fields.

19. The ultrasound imaging apparatus of claim 9, wherein
said image capturing unit comprises an emitting unit, an ultrasound probe, a receiving unit and a control unit, wherein said control unit is configured to control said emitting unit to emit signals to drive said ultrasound probe and to control said receiving unit to receive echo signals from said ultrasound probe;
said image processing unit comprises a processing unit, a compounding unit, and a scan and conversion unit, wherein said processing unit is configured to process the echo signals to produce the plurality of image signals;
said compounding unit is configured to compound the plurality of image signals; and
said scan and conversion unit is configured to scan and convert the compounded image data so as to be displayed.

20. The ultrasound imaging apparatus of claim 19, wherein said image capturing unit further comprises an operating unit for a user to enter instructions to said control unit.
